(19) United States
(12) Patent Application Publication
(10) Pub. No.: US 2006/0147913 A1
(43) Pub. Date: Jul. 6, 2006

(54) Method of analyzing substance on substrate by mass spectrometry

(76) Inventor: Tadashi Okamoto, Kanagawa (JP)

Correspondence Address: MORGAN & FINNEGAN, L.L.P., 3 WORLD FINANCIAL CENTER, NEW YORK, NY, 10281-2101, US

(21) Appl. No.: 10/518,559

(22) PCT Filed: Jun. 27, 2003
(86) PCT No.: PCT/JP03/08197

§ 371 Date: Dec. 21, 2004

(30) Foreign Application Priority Data
Jun. 28, 2002 (JP) 2002-191535

(51) Int. Cl. C12Q 1/68; G06F 19/00; G01N 33/48

(52) U.S. Cl. 435/6; 436/86; 702/19

(57) ABSTRACT

Provided is a method making it possible to measure the molecular weight useful for the specification of a substance bonded on a substrate by MALDI-TOF MS. When the substance is to be bonded on the substrate, MALDI-TOF MS analysis can be utilized by providing a partial structure to be disconnected by light in the bonded portion and selectively disconnecting the partial structure by light having a predetermined wavelength to bring the substance in an unfixed state.

### TECHNICAL FIELD

The present invention relates to a method of analyzing a substance fixed on a substrate and, more specifically, to a method of analyzing a plurality of bio-related substances fixed on a so-called biochip in a matrix form, a biochip on which bio-related substances are fixed in a way suitable for the application of the analytical method, and a method of analyzing a substance which interacts with the bio-related substance fixed on the biochip.

The present invention also relates to a method of determining a nucleic acid base sequence, and in particular, to a method of specifying the kind of one base added to a sequence primer during an extension reaction in base sequence analysis based on a dideoxy method, i.e., a method of determining a nucleic acid base sequence.

### BACKGROUND ART

A device having specific substances fixed on a substrate, particularly a so-called biochip having various probe molecules arranged on a substrate in a matrix form, such as a DNA chip or protein chip having a plurality of bio-related substances fixed in a matrix form has been used for the analysis of a genome or the analysis of gene expression. The results of analysis using those biochips are expected to provide important indices for the diagnosis of cancer, hereditary diseases, living habit diseases, infectious diseases, and the like, prognosis, the determination of a remedy, and the like.

There are known several methods of manufacturing a biochip. Taking a DNA chip as an example, typical DNA chip manufacturing methods include one in which photolithography is used to directly synthesize DNA probes on a substrate sequentially (U.S. Pat. No. 5,405,783) and one in which presynthesized DNA or cDNA (complementary DNA) is supplied and bonded to a substrate (U.S. Pat. No. 5,601,980, Japanese Patent Application Laid-Open No. 11-187900, Science Vol. 270, 467, 1995, etc.).

In general, a biochip is manufactured by one of the above methods. No matter which method is used to manufacture a biochip, when the biochip is to be used for the above purposes, it is very important that a probe existent on each matrix, that is, a bio-related substance in this case be a desired substance in order to ensure the reliability of analysis. If even a very small part of a substance existent on each matrix of a biochip is not a desired substance, a result obtained when the impurity functions as a probe molecule is included, thereby losing the reliability of analysis basically.

However, in an ordinary biochip manufacturing method including the above DNA chip manufacturing method, it cannot be always said that the possibility that an undesired substance is fixed at a specific position is completely eliminated. However, there has been unknown a method of specifying a substance fixed on a substrate. (The word “fixing” as used herein means a state where the substance is firmly bonded on a substrate like covalent bonding and not just adsorption.)

For instance, when time of flight type secondary ion mass spectrometry (to be abbreviated as “TOF-SIMS”) known as a highly sensitive surface analyzing technique is used, oligonucleotide formed on a gold substrate to a monomolecular film level can be analyzed (Proceeding of the twelfth International Conference on Secondary Ion Mass Spectrometry 951, 1999). However, the detected secondary ions are, for example, fragment ions such as P⁻, PO⁻, PO₂⁻ or PO₃⁻ obtained by segmenting the fixed substance. What kind of oligonucleotide the original substance is, that is, what the base sequence of oligonucleotide is cannot be known from the fragmented information. Although base fragment ions such as (adenine-H)⁻, (thymine-H)⁻, (guanine-H)⁻, (cytosine-H)⁻ and (uracil-H)⁻ are detected as other fragment ions, current TOF-SIMS analyzers do not have quantity determination accuracy and strictness high enough to identify the composition, for example, the whole base sequence without fail.

Another highly sensitive surface analyzing technique is X-ray photoelectron spectrometry (to be abbreviated as “XPS” hereinafter). Information obtained by this method relates to the composition of atoms or a bonding state between atoms. Information on the whole substance cannot be obtained by the method and the sensitivity of the method is not high enough to analyze nucleic acid of a monomolecular film level.

Meanwhile, as for the analysis of a substance adsorbed on a substrate, much attention is now paid to matrix-assisted laser desorption/ionization time-of-flight mass spectrometry (to be abbreviated as “MALDI-TOF MS”) in recent years as a method capable of analyzing the molecular weight of a substance with high sensitivity.

In MALDI-TOF MS, a substance which absorbs light having a specific wavelength and is called “matrix” and a specimen are mixed together and placed (adsorbed) on a stainless substrate for an analytical purpose, for example, and the substrate is irradiated with light which is absorbed by the matrix to desorb and ionize the specimen by energy transfer from the matrix to the specimen. The basic principle of MALDI-TOF MS is the mass spectrometry of the desorbing ions in terms of a time of flight. Fragment ions formed by primary ion irradiation are analyzed by the above TOF-SIM whereas the mass of even an unfragmented substance can be analyzed by MALDI-TOF MS. Therefore, when the target is nucleic acid, extremely important data on its base sequence can be obtained though the base sequence itself cannot be analyzed. At least when the measured mass of unfragmented nucleic acid differs from the targeted value, it is obvious that the nucleic acid does not have a desired base sequence.

However, since MALDI-TOF MS has a principle that a substance to be measured is not fragmented but desorbed as it is, a substance fixed on a substrate by covalent bonding, for example, cannot be desorbed and ionized as it is and therefore cannot be analyzed. That is, in the above biochip, a substance fixed on each matrix cannot be analyzed by MALTI-TOF MS as it is.

As means for solving the problem of MALDI-TOF MS, there is proposed a method of analyzing probe nucleic acid by arranging a bond decomposable with acidity at a specific position of probe nucleic acid and mixing it with a matrix and an acidic substance to disconnect and ionize the probe nucleic acid in order to analyze the multiple forms of a gene using a nucleic acid chip (Nucleic Acid Research, Vol. 29, No. 18, 3864, 2001). According to this method, the analysis of nucleic acid fixed on a substrate by covalent bonding is possible. Since the depurination of nucleic acid (DNA, RNA) occurs under an acidic condition and the nucleic acid is disconnected at the position of a purine base as described in the above document, the acidic condition cannot be strengthened, thereby deteriorating disconnection efficiency at the above targeted specific position and analytical sensitivity. In other words, when the acidic condition is strengthened to increase sensitivity, nucleic acid is disconnected at a position other than the above specific position.

### DISCLOSURE OF THE INVENTION

The inventor of the present invention have conducted studies on the above problem of the prior art and have made the following invention.

Namely, a method of analyzing a substance fixed on a substrate according to the present invention relates to a method of acquiring data on the mass of a substance fixed on a substrate, comprising:

selecting a structure including a partial structure to be disconnected by light to fix the substance on the substrate;

irradiating the substance fixed on the substrate with light for inducing the disconnection of the partial structure to be disconnected by light; and

analyzing the mass spectrum of the substance which is brought in an unfixed state by disconnecting the partial structure by the irradiation of light. A method of analyzing the mass spectrum preferably used is matrix assisted laser desorption/ionization time-of-flight mass spectrometry (to be abbreviated as MALDI-TOF MS). At that time, the light for inducing the disconnection of the partial structure to be disconnected by light is preferably a laser beam used for the analysis by MALDI-TOF MS. Also, the laser beam used for the analysis by MALDI-TOF MS may be a nitrogen laser beam having a wavelength of 337 nm. For example, the substance fixed on the substrate is preferably nucleic acid. The nucleic acid may be any one of DNA, RNA and PNA(peptide nucleic acid).

It is preferable to select a structure containing nitrobenzene as the partial structure to be disconnected by light. The structure containing nitrobenzene can be constructed with a compound represented by the following formula I.

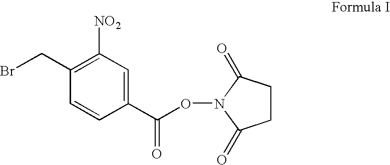

Also, the structure containing nitrobenzene can be constructed with a compound represented by the following formula II: 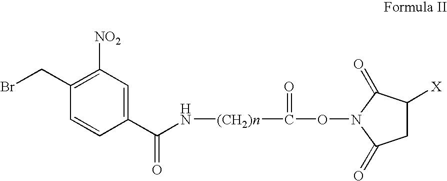
(wherein n is 3 or 4, and X is H or SO₃Na)

In addition, at that time, it is preferable that the substrate is a glass substrate having a primary amino group formed on the surface, a sulfanil (SH) group is bonded to the terminal of the substance, and the amino group and the sulfanil group are bonded together by a compound represented by the formula I or the formula II through a reaction between the amino group and the succinimide ester site of the compound and a reaction between the sulfanil group and the bromobenzyl site of the compound. Note that, the formation of a primary amino group on the glass substrate is preferably carried out by using a silane coupling agent having the primary amino group.

Alternatively, it is possible that the substrate is a glass substrate having a sulfanil group formed on the surface, an amino group is bonded to the terminal of the substance, and the sulfanil group and the amino group are bonded together by a compound represented by the formula I or the formula II through a reaction between the sulfanil group and the bromobenzyl site of the compound and a reaction between the amino group and the succinimide ester site of the compound. At that time, the formation of a sulfanil group on the glass substrate is preferably carried out by using a silane coupling agent having the sulfanil group.

Furthermore, the structure containing nitrobenzene may be constructed with a compound represented by the following formula III: 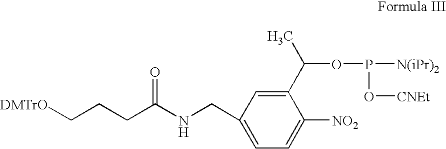
(wherein DMTrO is a dimethoxytrityloxy group and CNEt is a 2-cyanoethyl group).

In addition, a method of analyzing a biochip having a plurality of bio-related substances fixed on a substrate in a matrix form comprises:

selecting a structure including a partial structure to be disconnected by light to fix the bio-related substance on each matrix;

irradiating the bio-related substance fixed on the substrate with light for inducing the disconnection of the partial structure to be disconnected by light; and

analyzing the bio-related substance which has been brought in an unfixed state by disconnecting the partial structure through the irradiation of light by matrix-assisted laser desorption/ionization time-of-flight mass spectrometry. At that time, the light for inducing the disconnection of the partial structure to be disconnected by light is preferably a laser beam used for the analysis by MALDI-TOF MS. Also, the laser beam used for the analysis by MALDI-TOF MS may be a nitrogen laser beam having a wavelength of 337 nm. For example, the substance fixed on the substrate is preferably nucleic acid. The nucleic acid may be any one of DNA, RNA and PNA (peptide nucleic acid).

It is preferable to select a structure containing nitrobenzene as the partial structure to be disconnected by light. The structure containing nitrobenzene can be constructed with a compound represented by the following formula I.

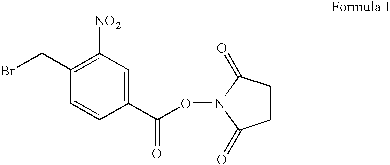

Also, the structure containing nitrobenzen can be with a compound represented by the following formula II: 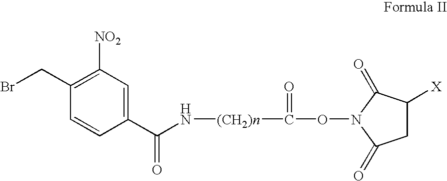
(wherein n is 3 or 4, and X is H or SO₃Na)

In addition, at that time, it is preferable that the substrate is a glass substrate having a primary amino group formed on the surface, a sulfanil (SH) group is bonded to the terminal of the substance, and the amino group and the sulfanil group are bonded together by a compound represented by the formula I or the formula II through a reaction between the amino group and the succinimide ester site of the compound and a reaction between the sulfanil group and the bromobenzyl site of the compound. Note that, the formation of a primary amino group on the glass substrate is preferably carried out by using a silane coupling agent having the primary amino group.

Alternatively, it is possible that the substrate is a glass substrate having a sulfanil group formed on the surface, an amino group is bonded to the terminal of the substance, and the sulfanil group and the amino group are bonded together by a compound represented by the formula I or the formula II through a reaction between the sulfanil group and the bromobenzyl site of the compound and a reaction between the amino group and the succinimide ester site of the compound. At that time, the formation of a sulfanil group on the glass substrate is preferably carried out by using a silane coupling agent having the sulfanil group.

Moreover, the structure containing nitrobenzene may be constructed with a compound represented by the following formula III: 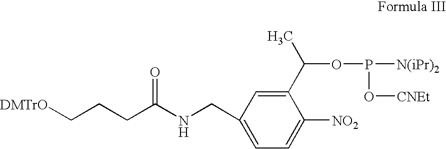
(wherein DMTrO is a dimethoxytrityloxy group and CNEt is a 2-cyanoethyl group).

According to the present invention, there is provided a biochip having a plurality of bio-related substances fixed on a substrate in a matrix form, characterized in that a structure including a partial structure to be disconnected by light is selected to fix the bio-related substances on each matrix. It is preferable that light for inducing the disconnection of the partial structure to be disconnected by light is a laser beam and, at that time, the laser beam is a nitrogen laser beam having a wavelength of 337 nm. For example, the bio-related substance fixed on the substrate is preferably nucleic acid. The nucleic acid may be any one of DNA, RNA and PNA (peptide nucleic acid).

It is preferable to select a structure containing nitrobenzene as the partial structure to be disconnected by light. The structure containing nitrobenzene is constructed with a compound represented by the following formula I.

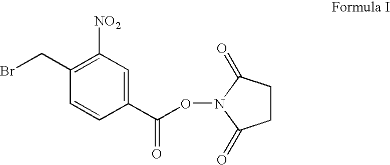

Also, the structure containing nitrobenzene can be constructed with a compound represented by the following formula II: 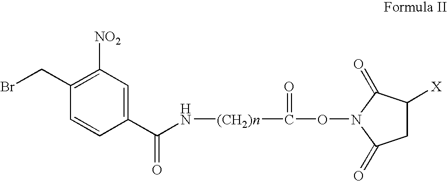
(wherein n is 3 or 4, and X is H or SO₃Na)

In addition, at that time, it is preferable that the substrate is a glass substrate having a primary amino group formed on the surface, a sulfanil (SH) group is bonded to the terminal of the substance, and the amino group and the sulfanil group are bonded together by a compound represented by the formula I or the formula II through a reaction between the amino group and the succinimide ester site of the compound and a reaction between the sulfanil group and the bromobenzyl site of the compound. Note that, the formation of a primary amino group on the glass substrate is preferably carried out by using a silane coupling agent having the primary amino group.

Alternatively, it is possible that the substrate is a glass substrate having a sulfanil group formed on the surface, an amino group is bonded to the terminal of the substance, and the sulfanil group and the amino group are bonded together by a compound represented by the formula I or the formula II through a reaction between the sulfanil group and the bromobenzyl site of the compound and a reaction between the amino group and the succinimide ester site of the compound. At that time, the formation of a sulfanil group on the glass substrate is preferably carried out by using a silane coupling agent having the sulfanil group.

Moreover, the structure containing nitrobenzene may be constructed with a compound represented by the following formula III: 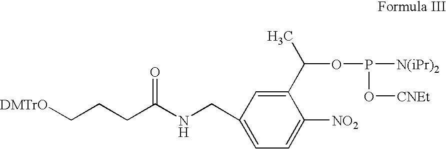
(wherein DMTrO is a dimethoxytrityloxy group and CNEt is a 2-cyanoethyl group).

Correspondingly, a method of analyzing a biochip having a plurality of bio-related substances fixed on a substrate in a matrix form comprises:

placing a substance which interacts with the bio-related substance on each matrix of the biochip under an interactive condition;

selecting a structure including a partial structure to be disconnected by light to fix the bio-related substance on each matrix;

irradiating the bio-related substance fixed on the substrate with light for inducing the disconnection of the partial structure to be disconnected by light; and

analyzing the bio-related substance which has been brought in an unfixed state by disconnecting the partial structure through the irradiation of light and the substance which has interacted with the bio-related substance at the same time by matrix-assisted laser desorpiton/ionization time-of-flight mass spectrometry. At that time, the light for inducing the disconnection of the partial structure to be disconnected by light is preferably a laser beam used for the analysis by MALDI-TOF MS. Note that, the laser beam used for the analysis by MALDI-TOF MS may be a nitrogen laser beam having a wavelength of 337 nm. For example, the bio-related substance fixed on the substrate is preferably nucleic acid. The nucleic acid may be any one of DNA, RNA and PNA (peptide nucleic acid).

It is preferable to select a structure containing nitrobenzene as the partial structure to be disconnected by the irradiation of light. The structure containing nitrobenzene can be constructed with a compound represented by the following formula I.

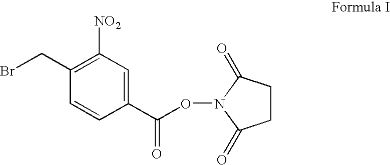

Also, the structure containing nitrobenzene can be constructed with a compound represented by the following formula II: 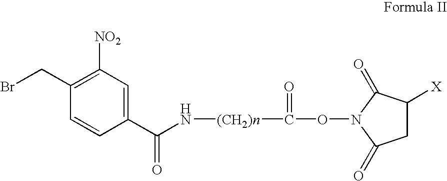
(wherein n is 3 or 4, and X is H or SO₃Na)

In addition, at that time, it is preferable that the substrate is a glass substrate having a primary amino group formed on the surface, a sulfanil (SH) group is bonded to the terminal of the substance, and the amino group and the sulfanil group are bonded together by a compound represented by the formula I or the formula II through a reaction between the amino group and the succinimide ester site of the compound and a reaction between the sulfanil group and the bromobenzyl site of the compound. Note that, the formation of a primary amino group on the glass substrate is preferably carried out by using a silane coupling agent having the primary amino group.

Alternatively, it is possible that the substrate is a glass substrate having a sulfanil group formed on the surface, an amino group is bonded to the terminal of the substance, and the sulfanil group and the amino group are bonded together by a compound represented by the formula I or the formula II through a reaction between the sulfanil group and the bromobenzyl site of the compound and a reaction between the amino group and the succinimide ester site of the compound. At that time, the formation of a sulfanil group on the glass substrate is preferably carried out by using a silane coupling agent having the sulfanil group.

Moreover, the structure containing nitrobenzene may be constructed with a compound represented by the following formula III: 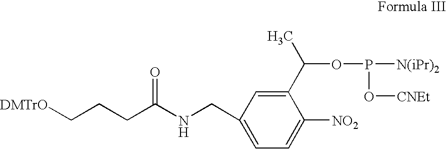
(wherein DMTrO is a dimethoxytrityloxy group and CNEt is a 2-cyanoethyl group).

Further, the present invention provides a biochip having bio-related substances fixed on a substrate, characterized in that the bio-related substances are fixed on the substrate by a partial structure to be disconnected by light.

The present invention makes it possible to analyze a substance bonded on a substrate by MALDI-TOF MS. It also makes it possible to analyze a nucleic acid probe bonded on a nucleic acid chip and targeted nucleic acid which forms a hybrid with the nucleic acid probe by MALDI-TOF MS.

### BEST MODE FOR CARRYING OUT THE INVENTION

The present invention is described in detail hereinbelow.

The present invention is characterized in that a substance fixed on a substrate by a structure including a partial structure to be disconnected by light is illuminated with light for disconnecting the partial structure and the above substance disconnected by the irradiation of light is analyzed by MALDI-TOF MS. The main point of the present invention is that the substrate is a so-called biochip having a plurality of bio-related substances fixed in a matrix form. It is a matter of course that the present invention is not limited to this biochip.

The present invention includes a biochip itself having a plurality of bio-related substances fixed on a substrate by a structure including a partial structure to be disconnected by light in order to enable the analysis of the bio-related substances by MALDI-TOF MS.

Further, the present invention is further characterized in that after a biochip having a plurality of bio-related substances fixed on a substrate by a structure including a partial structure to be disconnected by light and a substance which can interact with the bio-related substances are put under an interactive condition, the bio-related substances and the substance which can interact with the bio-related substances are analyzed by MALDI-TOF MS at the same time. In this case, interaction means the formation of a hybrid, interaction between an antibody and an antigen, interaction between a receptor and a ligand, or the like in nucleic acid.

In the present invention, when light to be irradiated at the time of analysis is a laser beam used for analysis by MALDI-TOF MS, that is, generally a nitrogen laser beam having a wavelength of 337 nm, disconnection by the irradiation of a laser beam and desorption/ionization occur at the same time in a MALDI-TOF MS apparatus during analysis, which is a desirable form. Another example of the laser beam used in this method is the second harmonic having a wavelength of 532 nm of an Nd: YAG laser. The problem to be encountered when a bond to be disconnected by acidity is used in the prior art is that nucleic acid is disconnected by depurination. Nucleic acid absorbs ultraviolet radiation (250 to 270 nm) to form a thymine dimmer in a case and may not cause desired interaction. In the above-mentioned laser wavelength range, there is no such an obstacle, which is desirable.

The substance fixed on the substrate to which the present invention is directed is not particularly limited. When the substrate is a so-called biochip, the substance may be nucleic acid such as DNA, RNA or PNA, or a nucleic acid analog which falls within the scope of the present invention.

The partial structure to be disconnected by light of the present invention is not limited but may be a structure containing nitrobenzene generally known as a structure which is optically cleaved. Since it is generally known that the structure containing nitrobenzene is cleaved by light having a wavelength of 350 to 400 nm, the above nitrogen laser may be preferably used as a light source.

The structure containing nitrobenzene can be constructed with a compound represented by the following formula I or formula II. 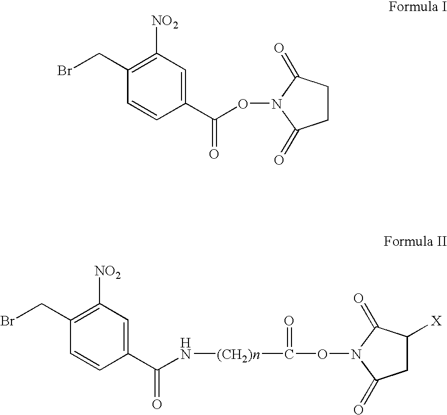
(wherein n is 3 or 4, and X is H or SO₃Na)

At that time, as the method of fixing a desired substance on a substrate may be used one in which a glass substrate having a primary amino group formed on the surface is used as the substrate, a sulfanil (SH) group is bonded to one end of the substance, and bonding between the amino group and the sulfanil group is carried out by a compound represented by the formula I or formula II, that is, a reaction between the amino group and the succinimido ester site of the compound and a reaction between the sulfanil group and the bromobenzyl site of the compound. In this case, the formation of a primary amino group on the glass substrate can be carried out by using a silane coupling agent having the primary amino group.

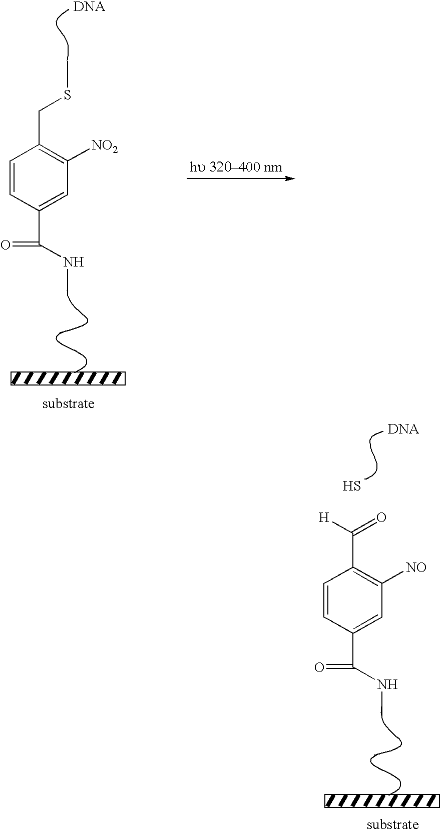

An example of the structure of nucleic acid bonded on the substrate, which is constructed by the above method is represented by the above chemical formula (left). An example of the structure disconnected by the irradiation of light is shown by the above chemical formula (right) (Biochemistry International Vol. 26, No. 5, 1992). As shown by the above examples, it is understood that the structure of the disconnected site of nucleic acid after disconnection is the same as the structure before disconnection. That is, DNA after cleavage returns to a state before bonding and the mass thereof is the same as that before bonding.

Japanese Patent Application Laid-Open No. 11-187900 discloses an example in which N-(6-maleimidecaproyloxy) succinimide represented by the following formula IV is used to bond nucleic acid having a sulfanil group to a substrate having a primary amino group formed thereon. In the present invention, desorption and analysis by MALDI-TOF MS are made possible by substituting this compound by the compound represented by the formula I or formula II.

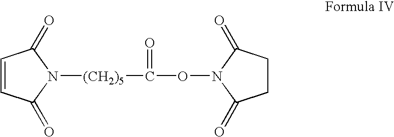

As another method of manufacturing a substrate, a glass substrate having a sulfanil group formed on the surface is used as the substrate, an amino group is bonded to one end of the substance, and bonding between the sulfanil group and the amino group is carried out by a compound represented by the formula I or formula II, that is, a reaction between the sulfanil group and the bromobenzyl site of the compound and a reaction between the amino group and the succinimido ester site of the compound. At that time, the formation of the sulfanil group on the glass substrate can be carried out by using a silane coupling agent having the sulfanil group.

The structure containing nitrobenzene can be constructed with a compound represented by the following formula III. However, this method is particularly effective when nucleic acid is bonded to a substrate. When probe nucleic acid is synthesized by a nucleic acid automatic synthesizer, for example, right before a unit having a functional group to be bonded to the substrate is introduced into the 3′ terminal or 5′ terminal of nucleic acid, a structure which can be optically cleaved can be introduced by the compound represented by the formula III. In this case, the functional group to be bonded to the substrate includes an amino group and a sulfanil group. A reagent for introducing the functional group in the nucleic acid automatic synthesizer is marketed by Glen Research Co., Ltd. and may be suitably used. As for a treatment on the substrate side, the above-described method using a silane coupling agent may also be used in this case. 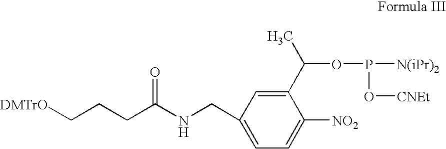
(wherein DMTrO is a dimethoxytrityloxy group, and CNEt is a 2-cyanoethyl group).

In MALDI-TOF MS, a substance called “matrix” is coexistent with a substance to be measured (specimen) to desorb and ionize the specimen as described above. The method which is frequently used is that the specimen is dissolved in a saturation solution of the matrix to a suitable concentration and, for example, several μl of the resulting co-existent solution is dropped on a stainless plate suitably given an address and dried so that the specimen is coexistent within the crystal of the matrix in a suitable concentration. In this case, a spectrum may not be obtained according to the crystal state and 3-D form of the matrix or the concentration of the specimen. Even if the spectrum is obtained, SN ratio or accuracy may not be satisfactory. When the surface of the crystal of the matrix is uneven or inclined even a little, as the specimen is existent in the matrix, the specimen may exert a bad influence upon the time of flight, whereby it may exert a bad influence upon the mass accuracy of the mass spectrum.

Since the specimen is fixed on the flat substrate in the present invention, the possibility that it exerts an influence upon the time of flight is relatively small. The supply of the matrix substance to the specimen can be carried out uniformly by applying the matrix to the substrate in a thin film form and the crystal state of the matrix becomes satisfactory, thereby making it possible to obtain a high mass accuracy. The application of the matrix substance may be suitably carried out by dipping, spin coating, or the like. When the thickness of the coating film is too large, the specimen is buried in the layer of the matrix substance, whereby the disconnection, dosorption and ionization of the specimen from the surface of the substrate may not be carried out completely and when the thickness is too small, the specimen is exposed from the matrix, whereby the disconnection, desorption and ionization of the specimen from the surface of the substrate may not be carried out completely. Therefore, the coating film must be thick enough and required for the disconnection, desorption and ionization of the specimen. In this case, the thickness of the coating film of the matrix substance large enough and required for the disconnection, desorption and ionization of the specimen is preferably 1 to 1,000 times the height of the specimen from the substrate. The thickness is not limited to this as a matter of course.

The following examples are provided to further illustrate the present invention.

Furthermore, the present invention relates to a method of determining a nucleic acid base sequence, In particular, the present invention also provides a method of determining a nucleic acid base sequence using matrix assisted laser desorption/ionization time-of-flight mass spectrometry for specifying the kind of one base added to a sequence primer due to an extension reaction in base sequence analysis based on a dideoxy method.

Hereinafter, a method of determining a nucleic acid sequence according to the present invention will be described in detail.

Under the international project, the operation of determining an entire base sequence of human genome DNA is advancing, and a draft base sequence of 90% or more has been published in 2000. Thus, the determination of gene information including a human gene is generally well-known as one of great results of the development of scientific technology in recent years.

Reading of a nucleic acid base sequence (i.e., sequencing) started in the beginning of 1970's, and went through the early Maxam-Gilbert method (Proceeding of the National Academy of Sciences, USA 74, 560, 1977). The sequencing currently in vogue is based on the dideoxy method (Proceeding of the National Academy of Sciences, USA 74, 5463, 1977) developed by Sanger et al.

Thereafter, the dideoxy method has been changed. Currently, a procedure is mainly used as sequencing, which is a combination of dye termination using 2′,3′-dideoxynuleotide triphosphate (ddNTP) of 4 kinds (i.e., adenosine, guanosine, cytidine, and uridine) labeled with different kinds, i.e., 4 kinds of fluorochromes and capillary electrophoresis. Hereinafter, this procedure will be described briefly.

(1) A part of a base sequence on a 3′-side from a site desired to be analyzed for the base sequence of nucleic acid (DNA) desired to be analyzed for the base sequence, or nucleic acid (DNA) complementary for the entire portion is annealed with nucleic acid desired for analysis of the base sequence, as a primer used for enzymatic nucleic acid extension reaction using nucleic acid desired to be analyzed for the base sequence as a template, whereby a hybrid is formed at the complementary base sequence portion.

(2) In the presence of appropriate amounts of 4 kinds of 2′-deoxynuleotide triphosphate (dNTP: N is A; adenine, G; guanine, C; cytosine, T; thymine) required for an enzymatic nucleic acid extension reaction and the above-mentioned 4 kinds of fluorescent labels 2′,3′-dideoxynucleotide triphosphate (ddNTP) as a terminator for an extension reaction, a base sequence from the sequence primer forming the above hybrid is subjected to an enzymatic extension reaction using desired nucleic acid as a template.

(3) The hybrid body subjected to the above extension reaction is dissociated to a single chain.

(4) The resultant solution containing an extension product of a plurality of chain lengths was subjected to chain length separation by capillary electrophoresis. The extended base is determined to be either of A, G, C, and T from fluorescence of the portion that has been extended by one base from the sequence primer, whereby the base sequence of a part of the extended portion or the remaining part is obtained.

(5) Based on the base sequence of the extended portion, a part or an entire part of the base sequence desired for analysis of nucleic acid which is desired for analysis of the base sequence is analyzed.

Recently, an apparatus has been developed in which, for example, 96 or 384 capillary bundles are used to complete the same number of electrophoresis in 2 to 3 hours. Further, regarding the operation of the same number of extension reactions and the like, automation using a robot has been attempted. Furthermore, a large amount of data can be analyzed at a high speed by using a high-performance computer. Consequently, the above-mentioned human entire genome analysis and the like also have been performed by a fluorescent dideoxy method and capillary electrophoresis.

However, regarding the base sequence analysis method based on the fluorescent dideoxy method and capillary electrophoresis used widely, the following problems also have been pointed out. That is:

one electrophoresis takes 2 to 3 hours;

it is necessary to exchange gel in a gel capillary for each electrophoresis;

a capillary is expensive, and it is necessary to exchange the capillary regularly;

the difference of one base is not necessarily read exactly. Therefore, electrophoresis needs to be performed a plurality of times for one extension product;

an enzymatic extension reaction needs to be performed separately; etc.

According to the present invention, in particular, new separation analysis means for solving the problems regarding electrophoresis will be proposed.

A method has been proposed in which a primer is fixed on a solid phase; an enzymatic extension reaction is effected; thereafter, an extension product is disconnected from the solid phase and subjected to mass spectrometry by a MALDI-TOF MS method, whereby sequencing is performed (Nucleic Acid Research Vol. 29, No. 18, 3864, 2001). According to this method, an N—P internucleotide bond capable of being subjected to acid decomposition is introduced to any nucleotide site extending from a linker; an acid material such as trifluoroacetate is mixed with an acid matrix; the above N—P bond is disconnected, and the disconnected fragment is analyzed. By using the dideoxy method in combination, base sequence information after the disconnection point can be obtained in principle. Furthermore, before being disconnected, the extension product including a primer portion is fixed on a solid phase. Therefore, it is relatively easy to remove impurities such as a template and a nucleotide monomer. However, this method has the following problems, and is different from the sequence determination method of the present invention.

More specifically, nucleic acid causes non-selective breakage at a purine site of adenosine and guanosine due to the phenomenon called depurination in an acid state. Therefore, the acid state during the above-mentioned N—P bond breakage cannot be strengthened. Consequently, breakage, desorption, and ionization efficiency are poor, so that sufficient sensitivity of mass spectrometry cannot be obtained. Furthermore, according to the method described in the above document, only one kind of primer is bonded to one substrate. Therefore, in order to simultaneously perform a plural number of times of sequencing of nucleic acid a plurality of times, an extension reaction is required for the corresponding number of substrates.

In contrast, according to the sequence determination method of the present invention, a primer is fixed on a solid phase, and an enzymatic extension reaction is effected. Thereafter, an extension product is disconnected from the solid phase, and subjected to mass spectrometry by a MALDI-TOF MS method, whereby sequencing is performed. When adopting this process, the extension product is disconnected from the solid phase by means capable of site-selectively disconnecting in a portion of a primer connected to the solid phase without using an acid material, instead of means for introducing an N—P internucleotide bond capable of being subjected to acid decomposition, and mixing an acid material such as trifluoroacetic acid to disconnect the N—P bond. Thus, desorption and ionization efficiency can be maintained by the MALDI-TOF MS method.

Furthermore, according to the sequence determination method of the present invention, as means capable of site-selectively disconnecting in a primer portion connected to a solid phase, and as means capable of site-selectively disconnecting in a connected portion of a primer with respect to the solid phase, a partial structure to be disconnected by light is provided on a 5′-side that does not influence hybridization with a template in the primer, and the partial structure to be disconnected by predetermined light irradiation after the extension reaction is selectively disconnected. Because of this, the conventional problem in which non-selective breakage is caused at a purine site of adenosine and guanosine due to the phenomenon called depurination in an acid state is avoided, and sequencing of nucleic acid with a very high operation efficiency can be performed.

That is, a method of determining a base sequence of nucleic acid according to the present invention is a method of determining a base sequence of nucleic acid, comprising the steps of:

(1) fixing, to a substrate, nucleic acid (DNA) complementary to a part or an entire part of a base sequence on a 3′-side from a site desired for analysis of a base sequence of nucleic acid (DNA) desired for analysis of the base sequence as a primer used for performing an enzymatic nucleic acid extension reaction, using the nucleic acid desired for analysis of the base sequence as a template, in a structure containing a partial structure to be disconnected by light on a 5′-side from the complimentary base sequence in the primer;

(2) annealing the nucleic acid desired for analysis of the base sequence to the primer fixed to the substrate at the complementary base sequence portion to form a hybrid;

(3) performing the enzymatic extension reaction using the nucleic acid desired for analysis of the base sequence as a template, on the substrate where the hybrid is formed, in the presence of appropriate amounts of 4 kinds of 2′-deoxynucleotide triphosphate (dNTP: N is A; adenine, G; guanine, C; cytosine, T; thymine) required for the enzymatic nucleic acid extension reaction and the 4 kinds of 2′,3′-dideoxynucleotide triphosphate (ddNTP) as a terminator for an extension reaction;

(4) removing the template nucleic acid from the substrate where the extension reaction is effected;

(5) irradiating a plurality of extension reaction products having different chain lengths including a primer portion fixed to the substrate in a structure containing a partial structure to be disconnected by light, with light for disconnecting the partial structure to be disconnected, analyzing a molecular weight of the extension product disconnected by the irradiation with light by a MALDI-TOF MS method, and clarifying a base sequence of an extension portion of the extension product based on an increase in a molecular weight from a molecular weight of the primer in the extension product; and

(6) analyzing a part or an entire part of the base sequence desired for analysis of nucleic acid desired for analysis of the base sequence, based on the base sequence of the extension portion.

It is preferable that in the process (5), the irradiation light is laser light used for analysis of the MALDI-TOF MS method. Further, it is preferable that the laser light used for analysis of the MALDI-TOF MS method is nitrogen laser light with a wavelength of 337 nm.

In the method of determining a base sequence of nucleic acid according to the present invention, in the process (5), a structure containing nitrobenzene may be selected as the partial structure to be disconnected by the irradiation with light. For example, the structure containing nitrobenzene may be structured with a compound represented by the above-mentioned formula I.

Further, the structure containing nitrobenzene may be structured using a compound represented by the above-mentioned formula II.

In such case, it is preferable that the substrate is a glass substrate on which a primary amino group is formed, a sulfanil (SH) group is bonded to a 5′-terminal of the primer, and the amino group is bonded to the sulfanil group via a compound represented by the formula I or a compound represented by the formula II by a reaction between the amino group and a succiimidoester site of the compound and a reaction between the sulfanil group and a bromobenzyl site of the compound. For example, the primary amino group may be formed on the glass substrate by using a silane coupling agent having a primary amino group. Alternately, it is possible that the substrate is a glass substrate on the surface of which a sulfanil group is formed, an amino group is bonded to a 5′-terminal of the primer, and the amino group is bonded to the sulfanil group via a compound represented by the formula I or a compound represented by the formula II by a reaction between the sulfanil group and bromobenzyl site of the compound and a reaction between the amino group and a succiimidoester site of the compound. In such case, for example, the sulfanil group may be formed on the glass substrate by using a silane coupling agent having a sulfanil group. Further, it is possible that the structure containing nitrobenzene is structured using a compound represented by the following formula III.

In the method of determining a base sequence of nucleic acid according to the present invention, it is preferable that enzyme used for the extension reaction has heat-resisting property. Furthermore, in particular, it is preferable that the substrate to which the primer is fixed is in a form of a nucleic acid chip in which a plurality of primer nucleic acids are placed in a matrix,

in the process (3), a part or an entire part of the primer nucleic acid is subjected to an enzymatic nucleic acid extension reaction together with the template thereof on the nucleic acid chip, and

in the process (4), the matrix portion subjected to the extension reaction is analyzed by the MALDI-TOF MS method.

According to the analysis method of a base sequence of the present invention, one analysis can be completed in several seconds to tens of seconds. Furthermore, for example, 384 kinds of samples are placed on one plate, and they can be analyzed automatically. Furthermore, tens of plates can be automatically analyzed simultaneously. Thus, tens of thousands of samples can be automatically measured overnight, which is very efficient in terms of time. Furthermore, a molecular weight can be obtained as an absolute value, so that ambiguity is eliminated, and a plate can be used a number of times.

More specifically, an extension product of nucleic acid as described above is analyzed by the method of the present invention, whereby sequencing of nucleic acid with a very high operation efficiency can be performed.

Furthermore, the extension product is a mixture of enzyme, each nucleotide monomer, each dye labeling terminator, a salt, and the like. In such a case, there is a problem in a method of efficient desorption/ionization of a desired extension product (Genomics Vol. 19, 417, 1994). Furthermore, in purifying an extension product, particularly, in sequencing of a number of nucleic acids, a great amount of labor is required.

According to the method of determining a base sequence of the present invention, an extension reaction product is disconnected from a substrate by irradiation with light. Therefore, the problem in the method of the above document, i.e., in the method of disconnecting using an acid material, that is, the influence of non-selective disconnection at a purine site of adenosine and guanosine due to the phenomenon called depurination can be substantially avoided.

At that time, if light to be irradiated is laser light used for analysis of the MALDI-TOF MS method, light irradiation for previous disconnection is not required to be performed prior to the analysis, and furthermore, disconnection, desorption, and ionization can be performed simultaneously, which is very convenient. Laser light used for the MALDI-TOF MS method is generally nitrogen laser light with a wavelength of 337 nm. However, there are several organic structures to be disconnected at this wavelength as described below. Thus, nitrogen laser can be used for the present invention. The present invention is not limited to laser light with this wavelength. For example, the second harmonic and the like with a wavelength of 532 nm such as Nd:YAG laser can be used. Nucleic acid is known to receive damage such as formation of a thymine dimer by irradiation with UV-light. The wavelength of UV-light causing formation of a thymine dimmer is in an absorption wavelength band of a nucleic acid sequence, i.e., about 250 to 270 nm. Thus, the above-mentioned laser wavelength used in the present invention can be used preferably without damaging nucleic acid.

A specific method for producing a substrate to which a primer is bonded used in the present invention is as follows. The above-mentioned substrate is a glass substrate on which a primary amino group is formed on a surface. A sulfanil (SH) group is bonded to a 5′-terminal of a primer. Bonding between the amino group and the sulfanil group is performed via a compound represented by the formula I or II, i.e., by a reaction between the amino group and a succiimidoester site and a reaction between the sulfanil group and a bromobenzyl site of the compound.

As described above, it is understood that the structure of a disconnected site of nucleic acid after disconnecting is the same as that before the disconnecting. More specifically, in this case, it is expected that during sequencing by the MALDI-TOF MS method, an increase by one base from an original molecular weight of a primer by the extension reaction is observed.

Another example of the substrate production method is as follows. The substrate is a glass substrate in which a sulfanil group is formed on a surface thereof. An amino group is bonded to a 5′-terminal of a primer. Bonding between the sulfanil group and the amino group is performed via the compound represented by the formula I or II, i.e., by a reaction between a sulfanil group and a bromobenzyl site of the compound, and a reaction between an amino group and a succiimidoester site of the compound. At this time, a sulfanil group is formed on a glass substrate by using a silane coupling agent having a sulfanil group.

Furthermore, another example of a disconnected containing nitrobenzene that can be used in the method of the present invention includes a structure structured by the above compound represented by the formula III. In this case, when primer nucleic acid is synthesized by a nucleic acid automatic synthesizer, immediately before a unit having a functional group to be bonded to a substrate is introduced to a 5′-terminal by the compound represented by the formula III, a structure capable of performing photofragmentation can be introduced. In this case, examples of the functional group to be bonded to the substrate include an amino group and a sulfanil group. A reagent for introducing such a functional group in a nucleic acid automatic synthesizer is available, for example, from Glen Research Corporation, so that they should be appropriately used. Furthermore, even in this case, the substrate side is treated by the above-mentioned method using a silane coupling agent.

As described above, according to the sequence determination method of the present invention, an enzymatic nucleic acid extension reaction is performed using, as a template, nucleic acid annealed with a primer fixed on a solid phase. At this time, it is efficient if annealing and then, an extension reaction can be performed in the presence of the enzyme used for the extension reaction, the substrate to which the primer is connected, or the template nucleic acid. During annealing, it is required to increase the temperature of the substrate with the primer connected thereto and the template nucleic acid to about 90° C. In this case, general enzyme cannot withstand such high temperature. In such a case, it is convenient to use heat-insulating enzyme. An example of such enzyme includes Thermo Sequenase DNA Polymerase (Amersham Pharmacia Biotech).

As one of the problems of the prior art, it has been described that the reaction such as an extension reaction needs to be separately conducted with respect to nucleic acid desired for sequencing. Furthermore, this also applies to the case where a primer is bonded to a solid phase, which has already been described.

As means for solving the problem, the second embodiment of the method of determining a base sequence of the present invention is characterized by adopting a method in which an enzymatic nucleic acid extension reaction is effected together with a template with respect to a part or an entire part of primer nucleic acid, on a so-called nucleic acid chip in which a plurality of primer nucleic acids are fixed in a matrix by using the above-mentioned means, and the matrix portion subjected to the extension reaction is analyzed by the above-mentioned MALDI-TOF MS method. At that time, if the base sequence of a primer is rendered a unique sequence with respect to each nucleic acid desired for sequencing contained in at least one extension reaction, each nucleic acid recognizes only a primer required for each extension reaction. Therefore, the extension reaction using all of the nucleic acids as a template can be performed once on one chip.

### EXAMPLE 1

Manufacture of nucleic acid bonded substrate by dT40 probe and analysis by MALDI-TOF MS

A substrate to which a nucleic acid probe was uniformly bonded was manufactured in accordance with the method disclosed by Japanese Patent Application Laid-Open NO. 11-187900. The differences between the above method and the method disclosed by the above publication are that a bifunctional crosslinking agent used to fix a nucleic acid primer was the compound represented by the following formula V which is one of compounds represented by the formula II that is, succinimidyl 6-(4-bromomethyl-3-nitrobenzoyl)aminohexanoate in place of N-(6-maleimidocaproyloxy)succinimide (compound represented by the Formula IV) and that a nucleic acid probe was bonded on the substrate uniformly and not in a matrix form. 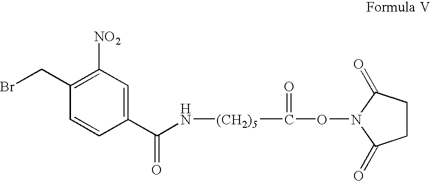
(1) Cleaning of Substrate

A synthetic quartz substrate measuring 25.4 mm×25.4 mm×1 mm was placed in a rack and immersed in an ultrasonic cleaner (Branson: GPIII) diluted with pure water to 10% for 24 hours. Thereafter, the substrate was subjected to ultrasonic cleaning in the cleaner for 20 minutes and rinsed to remove the cleaner. After it was rinsed in pure water, it was further subjected to an ultrasonic treatment in a container filled with pure water for 20 minutes. Then, the substrate was immersed in a 1N aqueous solution of sodium hydroxide heated at 80° C. in advance for 10 minutes. Subsequently, it was rinsed in water and pure water and the cleaned substrate was supplied to the next surface treatment step directly.

(2) Surface Treatment

A 1 wt % aqueous solution of N-β-(aminoethyl)-γ-aminopropyl trimethoxysilane KBM 603, silane coupling agent having an amino group bonded thereto (Shinetsu Chemical Co., Ltd.) was stirred at room temperature for 2 hours to hydrolyze the methoxy group in the molecule of the above silane compound. The cleaned substrate obtained in (1) was immersed in this solution at room temperature for 1 hour and rinsed in pure water, and a nitrogen gas was blown against both sides of the substrate to dry it. The substrate was baked in an oven heated at 120° C. for 1 hour to introduce the amino group onto the surface of the substrate in the end.

Thereafter, 5 mg of the compound represented by the Formula V (Dojin Kagaku Kenkyusho Co., Ltd.) was dissolved in a 1:1 (volume ratio) mixed solvent of dimethyl sulfoxide (DMS) and ethanol to a concentration of 0.5 mg/ml. A quartz substrate treated with a silane coupling agent was immersed in this compound represented by the Formula V solution at room temperature for 2 hours to react the amino group carried on the surface of the substrate by the silane coupling treatment with the succinimido group of the compound represented by the Formula V. In this stage, a bromoethyl group derived from the compound represented by the Formula V was existent on the surface of the substrate. The substrate lifted from the solution of the compound represented by the Formula V was cleaned with a DMSO/ethanol mixed solvent and ethanol sequentially, and a nitrogen gas was blown against the substrate to dry it.

(3) Synthesis of Probe DNA

A DNA manufacturer (Bex Co., Ltd.) was asked to synthesize singlestrand nucleic acid having sequence No. 1 (40-mer of dT). A sulfanil (SH) group was introduced into the 5′ terminal of the singlestrand DNA having sequence No. 1 by using a thiol modifier (Glen Research Co., Ltd.) at the time of synthesis. Deprotection and the collection of DNA were carried out by specified methods and HPLC was used for purification. The manufacturer was asked to carry out a series of steps from synthesis to purification. The calculated molecular weight of nucleic acid having sequence No. 1 was 12302.17 Da.

Sequence Number: 1

5′ HS-(CH₂)₆-O-PO₂-O-TTTTTTTTTT TTTTTTTTTT
TTTTTTTTTT TTTTTTTTTT 3′

(4) Bonding of DNA to Substrate

The singlestrand DNA having sequence No. 1 described in (3) was dissolved in a solution containing 7.5 wt % of glycerin, 7.5 wt % of urea, 7.5 wt % of thiodiglycol and 1 wt % of acetylene alcohol (trade name: Acetilenol EH; Kawaken Fine Chemical Co., Ltd.) to a concentration of 8 μM.

Thereafter, 25 μl of the above DNA solution was placed on the glass substrate whose surface was treated in (2) and covered with a 18 mm×18 mm cover glass sheet to react a bromoethyl group on the surface of the glass sheet with a sulfanil group at the terminal of the nucleic acid probe at room temperature. After 30 minutes, the substrate was rinsed in pure water and stored in pure water.

(5) Analysis by MALDI-TOF MS

After a nitrogen gas was blown against the nucleic acid chip manufactured in (4) to remove water, a suitable amount of the AG 50W-X8 ion exchange resin (BIO-RAD) was dispersed in 100 μl, and the dispersion was placed on a region including a portion to be analyzed of the surface of the chip and was left at room temperature for 5 minutes to carry out desalination.

Thereafter, the chip was cleaned with pure water to remove the ion exchange resin, water was removed by a nitrogen gas, and the chip was dried with a vacuum desiccator. This dried nucleic acid chip was analyzed by MALDI-TOF MS under the following conditions. During analysis, the substrate was fixed on a stainless plate for analysis by MALDI-TOF MS (Nippon Bulkar Daltonics) by a stainless pin and a stainless substrate holder.
Apparatus name: autoflex Reflectron (Japan Bruker Daltonics K. K.)
laser: nitrogen laser
acceleration voltage: 20 KV
measuring mode: linear mode
ionization: positive
internal standard: oligonucleotide; 6117, 9191 Da specification matrix: 3-hydroxy-2-propionic acid(3-HPA)
   (6) Analytical Results

The main peak was observed at a molecular weight of 12300.76 Da (Dalton). It is considered that the difference between this and a theoretical molecular weight of 12302.17 Da was due to a large difference in molecular weights between the used internal standard and the actually analyzed DNA. It can be understood from the results of this Example that a substance bonded on a substrate by covalent bonding in accordance with the method of the present invention can be analyzed by MALDI-TOF MS by selecting the bonded portion as the structure including a partial structure to be disconnected by light.

### COMPARATIVE EXAMPLE 1

Bonding of DNA by compound represented by the Formula IV and trial analysis by MALDI-TOF MS

A DNA bonded substrate was manufactured by the same procedure and under the same conditions as those in Example 1 except that compound represented by the Formula IV which could not form a partial structure to be disconnected by light was used in place of the compound represented by the Formula V used for fixing to the substrate. An attempt was made to analyze this DNA bonded substrate by MALDI-TOF MS under exactly the same measurement conditions as those in Example 1 but a clear peak was not observed.

### EXAMPLE 2

Manufacture of DNA Chip and Analysis by MALDI-TOF MS

(1) Manufacture of DNA Chip

A Teflon print-slide glass sheet having 30 black Teflon resin wells (diameter of 2 mm) formed on a slide glass sheet (ER-212: Funakoshi Pharmaceutical Co., Ltd.) was used as the substrate to carry out an UV-ozone treatment, followed by the cleaning and surface treatment of Example 1.

Sequence Number: 2

5′ HS-(CH₂)₆-O-PO₂-O-ACTGGCCGTCGTTTTACA 3′

Thereafter, DNA having sequence No. 2 was dissolved in the same manner as in Example 1 and the resulting solution was supplied to the wells of the slide glass sheet subjected to the above surface treatment in an amount of 4 μl for each well and the whole was left in a humidity maintained chamber at room temperature for 30 minutes to react DNA with the substrate. Then, DNA was cleaned with pure water and stored in pure water. The calculated molecular weight of nucleic acid having sequence No. 2 was 5661.84.

(2) Analysis by MALDI-TOF MS

The DNA chip manufactured in (1) was analyzed by MALDI-TOF MS in the same manner as that in Example 1 except that the supply of an ion exchange resin dispersion to each well was 4 μl. Analysis was carried out by irradiating the inside of each well with a laser beam spot. The molecular weights of the used internal standard nucleic acids were 3645 Da and 6117 Da.

(3) Analytical Results

The main peak was observed at a molecular weight of 5662.05 Da. It was found from the results of this Example that a DNA probe bonded on a DNA chip can be analyzed by MALDI-TOF MS by selecting the bonded portion as the structure including a partial structure to be disconnected by light when the DNA probe was bonded on the substrate by the method of the present invention.

### EXAMPLE 3

Hybridization on the DNA chip and analysis by MALDI-TOF MS

(1) Hybridization

| | |
|---|---|
| 5′ TGTAAAACGACGGCCAGT 3′ | Sequence Number: 3 |

DNA having sequence No. 3 which is complementary to the base sequence (sequence number: 2) of the probe nucleic acid of the DNA chip manufactured in Example 2 was synthesized. This DNA was dissolved in a 50 mM phosphoric acid buffer solution (pH=7.0) containing 1 M of NaCl to a concentration of 50 pM. Then, 5 μl of this DNA solution was supplied to each well of the DNA chip manufactured in Example 2 and the chip was covered with a cover glass sheet to carry out hybridization in a humidity maintained chamber at 45° C. for 15 hours. Then, the DNA chip was cleaned with cold pure water for 30 seconds, pure water was removed with a nitrogen gas, and then the DNA chip was dried in a desiccator. The calculated molecular weight of nucleic acid having sequence No. 3 was 5532.07 Da.

(2) Analysis by MALDI-TOF MD

Analysis was carried out by MALDI-TOF MS in completely the same manner as in Example 2 except that desalination was not carried out.

(3) Analytical Results

Two peaks were observed at molecular weights of 5662.05 Da and 5532.57 Da. They were considered to be derived from a DNA probe having sequence No. 2 bonded on the DNA chip and DNA having sequence No. 3 forming a hybrid with the probe.

It was found from the results of this Example that the DNA probe bonded on the DNA chip and the target DNA forming a hybrid with the DNA probe can be analyzed by MALDI-TOF MS by selecting the bonded portion as the structure including a partial structure to be disconnected by light when the DNA probe is bonded on the substrate by the method of the present invention.

### EXAMPLE 4

Manufacture of nucleic acid bonded substrate by dT 40 primer

A substrate to which a nucleic acid primer was uniformly bonded was manufactured in accordance with the method disclosed by Japanese Patent Application Laid-Open NO. 11-187900. The differences between the above method and the method disclosed by the above publication are that a bifunctional crosslinking agent used to fix a nucleic acid primer was the compound represented by the following formula V which is one of compounds represented by the formula II that is, succinimidyl 6-(4-bromomethyl-3-nitrobenzoyl)aminohexanoate in place of N-(6-maleimidocaproyloxy)succinimide (compound represented by the formula IV) and that a nucleic acid primer was bonded on the substrate uniformly and not in a matrix form. 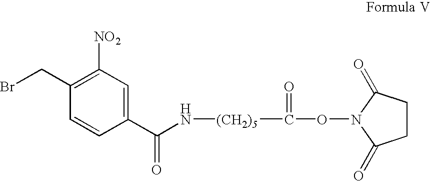
(1) Cleaning of Substrate

A synthetic quartz substrate measuring 25.4 mm×25.4 mm×1 mm was placed in a rack and immersed in an ultrasonic cleaner (Branson: GPIII) diluted with pure water to 10% for 24 hours. Thereafter, the substrate was subjected to ultrasonic cleaning in the cleaner for 20 minutes and rinsed to remove the cleaner. After it was rinsed in pure water, it was further subjected to an ultrasonic treatment in a container filled with pure water for 20 minutes. Then, the substrate was immersed in a 1N aqueous solution of sodium hydroxide heated at 80° C. in advance for 10 minutes. Subsequently, it was rinsed in water and pure water and the cleaned substrate was subjected to the next surface treatment step directly.

(2) Surface Treatment

A 1 wt % aqueous solution of N-β-(aminoethyl)-γ-aminopropyl trimethoxysilane KBM 603, silane coupling agent having an amino group bonded thereto (Shinetsu Chemical Co., Ltd.) was stirred at room temperature for 2 hours to hydrolyze the methoxy group in the molecule of the above silane compound. The cleaned substrate obtained in (1) was immersed in this solution at room temperature for 1 hour and rinsed in pure water, and a nitrogen gas was blown against both sides of the substrate to dry it. The substrate was baked in an oven heated at 120° C. for 1 hour to introduce the amino group onto the surface of the substrate in the end.

Thereafter, 5 mg of the compound represented by the formula V (Dojin Kagaku Kenkyusho Co., Ltd.) was dissolved in a 1:1 (volume ratio) mixed solvent of dimethyl sulfoxide (DMSO) and ethanol to a concentration of 0.5 mg/ml. A quartz substrate treated with a silane coupling agent was immersed in this compound represented by the formula V solution at room temperature for 2 hours to react the amino group carried on the surface of the substrate by the silane coupling treatment with the succinimido group of the compound represented by the formula V. In this stage, a bromoethyl group derived from the compound represented by the formula V was existent on the surface of the substrate. The substrate lifted from the solution of the compound represented by the formula V was cleaned with a DMSO/ethanol mixed solvent and ethanol sequentially, and a nitrogen gas was blown against the substrate to dry it.

(3) Synthesis of Probe DNA

A DNA manufacturer (Bex Co., Ltd.) was asked to synthesize singlestrand nucleic acid having sequence No. 1 (40-mer of dT). A sulfanil (SH) group was introduced into the 5′ terminal of the singlestrand DNA having sequence No. 1 by using a thiol modifier (Glen Research Co., Ltd.) at the time of synthesis. Deprotection and the collection of DNA were carried out by specified methods and HPLC was used for purification. The manufacturer was asked to carry out a series of steps from synthesis to purification. The calculated molecular weight of nucleic acid having sequence No. 1 was 12302.17 Da.

Sequence Number: 1

5′ HS-(CH₂)₆-O-PO₂-O-TTTTTTTTTT TTTTTTTTTT
TTTTTTTTTT TTTTTTTTTT 3′

(4) Bonding of DNA to Substrate

The singlestrand DNA having sequence No. 1 described in (3) was dissolved in a solution containing 7.5 wt % of glycerin, 7.5 wt % of urea, 7.5 wt % of thiodiglycol and 1 wt % of acetylene alcohol (trade name: Acetilenol EH; Kawaken Fine Chemical Co., Ltd.) to a concentration of 8 μM.

Thereafter, 25 μl of the above DNA solution was placed on the glass substrate whose surface was treated in (2) and covered with a 18 mm×18 mm cover glass sheet to react a bromoethyl group on the surface of the glass sheet with a sulfanil group at the terminal of the nucleic acid probe at room temperature. After 30 minutes, the substrate was rinsed in pure water and stored in pure water.

### EXAMPLE 5

Extension Reaction and Analysis by MALDI-TOF MS

(1) Extension Reaction

Previously, DNA polymerase for DNA sequencing (Thermo Sequenase: Amersham Pharmacia Biotech) is diluted to 1 unit/μl with an attached buffer for Then, 4.5 μl of a reaction buffer attached to Thermo Sequenase and 5 μl of Poly(dA) solution (Amersham Pharmacia Biotech: average chain length 300; dissolved in pure water in a concentration of 0.25 μg/μl) were taken to 0.5 ml of an Eppendorf tube. Then, a deoxyribonucleotide mixed solution of 4 kinds (Sequencing Grade Solution dNTPs: Amersham Pharmacia Biotech; 100 mM) and 1 μl of dideoxyribonucleotide mixed solution of 4 kinds (Sequencing Grade Solution ddNTPs: Amersham Pharmacia Biotech; 5 mM) are added to the Eppendorf tube. Pure water is further added thereto to adjust the total capacity to 43 μl.

To this solution, 2 μl of a Thermo Sequenase solution previously diluted is added, followed by mixing thoroughly. Thereafter, 20 μl of the resultant mixture is poured to a well in an incubation chamber for hybridization (CoverWell 20 μl Chanber: Funakoshi Co., Ltd.). A substrate with DNA bonded thereto prepared in Example 1 is placed on the well so that the treated side is brought into contact with the liquid. The substrate is tightly brought in to contact with the liquid so that the liquid does not leak. The chamber was placed on a hot plate whose temperature is adjustable, and heated at 85° C. for 10 minutes. Then, the chamber was allowed to stand in an incubator at 50° C. for 30 minutes, whereby completion of the reaction, the substrate was peeled from the chamber, washed with pure water at 85° C. for 2 minutes, and rinsed with flowing pure water for one minute, whereby template DNA, an unreacted monomer, and the like were washed from the substrate. Then, nitrogen gas was sprayed onto the substrate so as to remove water. Thereafter, an appropriate amount of ion exchange resin AG 50W-X8 (BIO-RAD) was dispersed in 100 μl and placed on a region containing a portion to be analyzed on the chip surface. The substrate was allowed to stand at room temperature for 5 minutes, and desalination was performed. Then, the substrate was washed with pure water, water was removed by nitrogen gas, and stored in a vacuum desiccator.

(2) Analysis by MALDI-TOF MS

The substrate subjected to the extension reaction was analyzed by MALDI-TOF MS under the following condition. During analysis, the substrate was fixed to a stainless plate (Bruker Daltonics (Japan) Inc.) for MALDI-TOF MS analysis, using a stainless pin and a substrate holder made of stainless steel.
Apparatus name: autoflex Reflectron (Bruker Daltonics (Japan) Inc.)
Laser: Nitrogen laser
Acceleration voltage: 20 KV
Measurement mode: linear mode
Ionization: positive
Internal standard: oligonucleotide; 6117, 9191 Da
Specification matrix: 3-hydroxy-2-propionic acid (3-HPA)
   (3) Analysis Result

A mass spectrum was observed in which the molecular weight of 304.19 Da was increased successively with the molecular weight 12588.95 Da being the minimum. The calculated value of a molecular weight in the case where DNA of SEQ ID NO: 1 was disconnected by irradiation with light is 12302.17, and the calculated value of a molecular weight in the case where ddTTP was bonded thereto (bonded portion is simple phosphoric diester) is 12590.36 Da. When dNTP is added successively before terminated with ddTTP, the calculated value of the addition of a molecular weight is 304.19 Da. Thus, at an observed peak where the molecular weight is increased successively, it is considered that dNTP is increased by one molecule. It is known from this that template DNA whose base sequence is attempted to obtain as a model is a continuous sequence of adenosine. More specifically, it is understood that the base sequence of nucleic acid can be analyzed by the method of the present invention.

The reason why the observed absolute value of the molecular weight is shifted from the theoretical molecular weight is considered as follows: since the difference in molecular weight between the internal standard used for calibrating a mass and actually analyzed DNA is large, a systematic shift is caused in a measurement value.

### EXAMPLE 6

Analysis of phage DNA base sequence by MALDI-TOF MS

(1) Manufacture of DNA Chip

A Teflon print slide glass sheet having 30 black Teflon resin wells (diameter of 2 mm) formed on a slide glass sheet (ER-212: Funakoshi Pharmaceutical Co., Ltd.) was used as the substrate to carry out an UV-ozone treatment, followed by the cleaning and surface treatment of Example 1.

Sequence Number: 4

5′ HS-(CH₂)₆-O-PO₂-O-TGTAAAACGACGGCCAGT 3′

Next, DNA of SEQ ID NO: 2 was dissolved in the same way as in Example 1, and supplied by 4 μl to a well of the slide glass subjected to the above surface treatment. The well was allowed to stand in a moisture-retention chamber at room temperature for 30 minutes, whereby DNA was reacted with the substrate. Then, the substrate was washed with pure water, and stored in pure water. Nucleic acid of SEQ ID NO: 2 is a base sequence complementary to a base sequence of 6318 to 6301 of single stranded (+strand) bacteriophage M13, and the calculated value of a molecular weight is 5728.84.

(2) Extension Reaction

The extension reaction and aftertreatment were performed under the same condition, except that the template DNA of Example 2 was set to be 2.5 μg of M13mp18(+)Strand DNA (Amersham Pharmacia Biotech). Furthermore, desalination was performed with respect to each well.

(3) Analysis by MALDI-TOF MS

A DNA chip subjected to the extension reaction was analyzed by MALDI-TOF MS. The analysis was performed by irradiating the inside of the well with a laser spot. Furthermore, internal standard nucleic acid used for the analysis is 3645 Da, 6117 Da, 9191 Da.

(4) Analysis Result

A mass spectrum was observed in which the molecular weight was increased successively with the molecular weight 6042.10 Da being the minimum. The calculated value of a molecular weight in the case where DNA of SEQ ID NO: 2 was bonded to ddGTP is 6042-05 Da. Therefore, it is understood that what was first added to a primer was G, i.e., the sequence of C in a template. Similarly, analysis was successively continued based on an increase in a molecular weight, and the base sequence of a template was analyzed to about 180th base from the first C: CGGTTCGAAC GTACGGACGT CCAGCTGAGA TCTCCTAGGG GCCCATGGCT CGAGCTTAAG . . . . The analyzed base sequence was perfectly matched with the base sequence described in the document. Because of this, it is understood that the base sequence of a template can be analyzed with a primer chip according to the method of the present invention.

### EXAMPLE 7

Simultaneous analysis of Poly(dA), phage DNA base sequence

A DNA chip with two kinds of primers used in Examples 2 and 3 placed on different matrixes on the chip was produced by the same method as that of Example 3. Then, an extension reaction was effected in the presence of Poly(dA) used in Example 2 and M13mp18(+)Strand DNA used in Example 3, and each matrix was analyzed by MALDI-TOF MS in accordance with the method described above. As a result, the same base sequence information as those of Examples 2 and 3 was obtained from each matrix. That is, it is understood that a primer unique to template nucleic acid of interest is placed on each matrix of a primer chip and subjected to one extension reaction, whereby the base sequence can be analyzed in parallel with respect to each template.

1. A method of acquiring data on the mass of a substance fixed on a substrate, comprising the steps of:
using a structure including a partial structure to be disconnected by light to fix the substance on the substrate;
irradiating the substance fixed on the substrate with light for inducing the disconnection of the partial structure to be disconnected by light; and
analyzing the mass spectrum of the substance which is brought in an unfixed state by disconnecting the partial structure by the irradiation of light.

2. The method according to claim 1, wherein means of analyzing the mass spectrum is matrix assisted laser desorption ionization time-of-flight mass spectrometry (to be abbreviated as MALDI-TOF MS).

3. The method according to claim 2, wherein light for inducing the disconnection of the partial structure to be disconnected by light is a laser beam used for the analysis by MALDI-TOF MS.

4. The method according to claim 3, wherein the laser beam used for the analysis by MALDI-TOF MS is a nitrogen laser beam.

5. The method according to claim 1, wherein the substance fixed on the substrate is nucleic acid.

6. The method according to claim 1, wherein a structure containing nitrobenzene is selected as the partial structure to be disconnected by the irradiation of light.

7. The method according to claim 6, wherein the structure containing nitrobenzene is constructed with a compound represented by the following formula I or II: 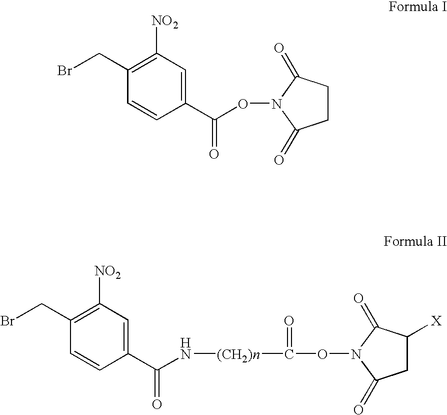
(wherein n is 3 or 4, and X is H or SO₃Na).

8. The method according to claim 7, wherein the substrate is a glass substrate having a primary amino group formed on the surface, a thiol (SH) group is bonded to the terminal of the substance, and the amino group and the thiol group are bonded together by a compound represented by the formula I or the formula II through a reaction between the amino group and the succinimide ester site of the compound and a reaction between the thiol group and the bromobenzyl site of the compound.

9. The method according to claim 8, wherein the formation of a primary amino group on the glass substrate is carried out by using a silane coupling agent having the primary amino group.

10. The method according to claim 7, wherein the substrate is a glass substrate having a sulfanil group formed on the surface, an amino group is bonded to the terminal of the substance, and the thiol group and the amino group are bonded together by a compound represented by the formula I or the formula II through a reaction between the thiol group and the bromobenzyl site of the compound and a reaction between the amino group and the succinimide ester site of the compound.

11. The method according to claim 10, wherein the formation of a thiol group on the glass substrate is carried out by using a silane coupling agent having the thiol group.

12. The method according to claim 6, wherein the structure containing nitrobenzene is constructed with a compound represented by the following formula III: 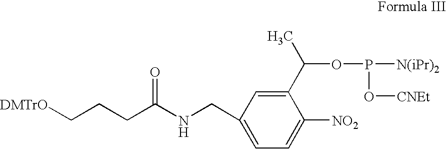
(wherein DMTrO is a dimethoxytrityloxy group and CNEt is a 2-cyanoethyl group).

13. The method according to claim 2, wherein a substance (matrix substance) for assisting the desorption and ionization of the substance fixed on the substrate is applied to at least a region to be used for the mass spectrometry of the substrate.

14. The method according to claim 13, wherein the thickness of the coating film of the matrix substance is large enough and required for the desorption and ionization of the substance fixed on the substrate.

15. A method of acquiring data on the mass of a bio-related substance on each matrix of a biochip having a plurality of bio-related substances fixed on a substrate in a matrix form by a structure including a partial structure to be disconnected by light, the method comprising the steps of:
irradiating the bio-related substance on each matrix fixed on the substrate with light for inducing the disconnection of the partial structure to be disconnected by light; and
analyzing the mass spectrum of the bio-related substance which is brought in an unfixed state by disconnecting the partial structure by the irradiation of light.

16. The biochip having a plurality of bio-related substances fixed on a substrate in a matrix form, wherein the bio-related substance is fixed by a partial structure to be disconnected by light.

17. The biochip according to claim 16, wherein the bio-related substance fixed on the substrate is nucleic acid.

18. The biochip according to claim 17, wherein the nucleic acid is DNA.

19. The biochip according to claim 17, wherein the nucleic acid is RNA.

20. The biochip according to claim 17, wherein the nucleic acid is PNA (peptide nucleic acid).

21. The biochip according to claim 16, wherein the partial structure to be disconnected by the irradiation of light has a structure containing nitrobenzene.

22. The biochip according to claim 21, wherein the structure containing nitrobenzene is constructed with a compound represented by the following formula I or II: 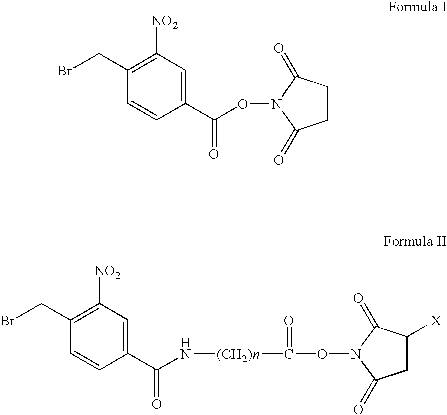
(wherein n is 3 or 4, and X is H or SO₃Na).

23. The biochip according to claim 21, wherein the structure containing nitrobenzene is constructed with a compound represented by the following formula III: 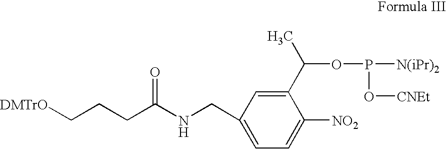
(wherein DMTrO is a dimethoxytrityloxy group and CNEt is a 2-cyanoethyl group).

24. A method of acquiring data on the mass of a bio-related substance on each matrix of a biochip having a plurality of bio-related substances fixed on a substrate in a matrix form and the mass of a substance which interacts with the bio-related substance, the method comprising the steps of:
fixing the bio-related substance on each matrix on the substrate by a structure including a partial structure to be disconnected by light;
placing the substance which interacts with the bio-related substance on each matrix of the biochip under an interactive condition;
irradiating the bio-related substance fixed on the substrate with light for inducing the disconnection of the partial structure to be disconnected by light; and
analyzing the mass spectra of the bio-related substance which has been brought in an unfixed state by the irradiation of light and the substance which has interacted with the bio-related substance in an unfixed state at the same time by disconnecting the partial structure.

25. An MALDI-TOF MS apparatus comprising:
means of moving a biochip to which bio-related substances are fixed with a structure to be disconnected by light to a position for analysis from a predetermined position; and
means of sequentially analyzing substances on each matrix in a specified order based on information on the shape of the biochip and the arrangement of matrices on the biochip at the position for analysis and moving the substances on each matrix from the position for analysis to the predetermined position.

26. A biochip having bio-related substances fixed on a substrate, wherein the bio-related substances are fixed on the substrate by a partial structure to be disconnected by light.

27. A method of determining a base sequence of nucleic acid, comprising the steps of:
(1) fixing, to a substrate, nucleic acid (DNA) complementary to a part or an entire part of a base sequence on a 3′-side from a site desired for analysis of a base sequence of nucleic acid (DNA) desired for analysis of the base sequence as a primer used for performing an enzymatic nucleic acid extension reaction, using the nucleic acid desired for analysis of the base sequence as a template, in a structure containing a partial structure to be disconnected by light on a 5′-side from the complimentary base sequence in the primer;
(2) annealing the nucleic acid desired for analysis of the base sequence to the primer fixed to the substrate at the complementary base sequence portion to form a hybrid;
(3) performing the enzymatic extension reaction using the nucleic acid desired for analysis of the base sequence as a template, on the substrate where the hybrid is formed, in the presence of appropriate amounts of 4 kinds of 2′-deoxynucleotide triphosphate (dNTP: N is A; adenine, G; guanine, C; cytosine, T; thymine) required for the enzymatic nucleic acid extension reaction and the 4 kinds of 2′,3′-dideoxynucleotide triphosphate (ddNTP) as a terminator for an extension reaction;
(4) removing the template nucleic acid from the substrate where the extension reaction is effected;
(5) irradiating a plurality of extension reaction products having different chain lengths including a primer portion fixed to the substrate in a structure containing a partial structure to be disconnected by light, with light for disconnecting the partial structure to be disconnected, analyzing a molecular weight of the extension product disconnected by the irradiation with light by a MALDI-TOF MS method, and clarifying a base sequence of an extension portion of the extension product based on an increase in a molecular weight from a molecular weight of the primer in the extension product; and
(6) analyzing a part or an entire part of the base sequence desired for analysis of nucleic acid desired for analysis of the base sequence, based on the base sequence of the extension portion.

28. The method according to claim 27, wherein in the process (5), the irradiation light is laser light used for analysis of the MALDI-TOF MS method.

29. The method according to claim 27, wherein the laser light used for analysis of the MALDI-TOF MS method is nitrogen laser light with a wavelength of 337 nm.

30. The method according to claim 27, wherein in the process (5), a structure containing nitrobenzene is selected as the partial structure to be disconnected by the irradiation with light.

31. The method according to claim 27, wherein the structure containing nitrobenzene is structured using a compound represented by the following formula I or II: 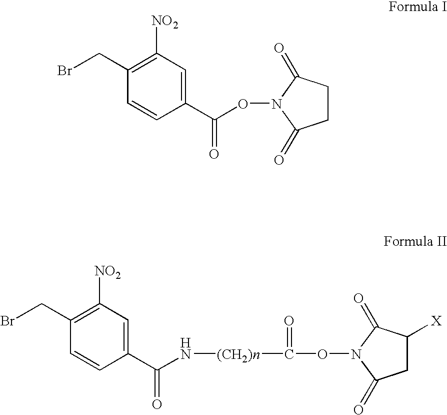
(where, n=3 or 4, X=H or SO₃Na).

32. The method according to claim 31, wherein the substrate is a glass substrate on the surface of which a primary amino group is formed, a sulfanil (SH) group is bonded to a 5′-terminal of the primer, and the amino group is bonded to the sulfanil group via a compound represented by the formula I or a compound represented by the formula II by a reaction between the amino group and a succiimidoester site of the compound and a reaction between the sulfanil group and a bromobenzyl site of the compound.

33. The method according to claim 32, wherein the primary amino group is formed on the glass substrate by using a silane coupling agent having a primary amino group.

34. The method according to claim 31, wherein the substrate is a glass substrate on the surface of which a sulfanil group is formed, an amino group is bonded to a 5′-terminal of the primer, and the amino group is bonded to the sulfanil group via a compound represented by the formula I or a compound represented by the formula II by a reaction between the sulfanil group and bromobenzyl site of the compound and a reaction between the amino group and a succiimidoester site of the compound.

35. The method according to claim 34, wherein the sulfanil group is formed on the glass substrate by using a silane coupling agent having a sulfanil group.

36. The method according to claim 30, wherein the structure containing nitrobenzene is structured using a compound represented by the following formula III: 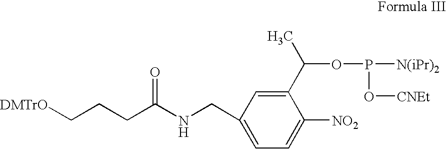
(wherein DMTrO is a dimethoxytrityloxy group and CNEt is a 2-cyanoethyl group).

37. The method according to claim 27, wherein enzyme used for the extension reaction has heat resisting property.

38. The method according to claim 27, wherein the substrate to which the primer is fixed is in a form of a nucleic acid chip in which a plurality of primer nucleic acids are placed in a matrix,
in the process (3), a part or an entire part of the primer nucleic acid is subjected to an enzymatic nucleic acid extension reaction together with the template thereof on the nucleic acid chip, and
in the process (4), the matrix portion subjected to the extension reaction is analyzed by the MALDI-TOF MS method.


SEQUENCE LISTING (4 sequences)

SEQ ID NO: 1 - DNA, 40 bp, Artificial
  Probe
tttttttttttttttttttttttttttttttttttttttt

SEQ ID NO: 2 - DNA, 18 bp, Artificial
  Probe
actggccgtcgttttaca

SEQ ID NO: 3 - DNA, 18 bp, Artificial
  Probe
tgtaaaacgacggccagt

SEQ ID NO: 4 - DNA, 18 bp, Artificial
  Probe
tgtaaaacgacggccagt